(19) United States
(12) Patent Application Publication
(10) Pub. No.: US 2015/0164629 A1
(43) Pub. Date: Jun. 18, 2015

(54) SURGICAL METHODS AND DEVICES FOR TREATMENT OF PROLAPSED UTERUS

(71) Applicant: Carey Tasca Pty Ltd, Elthem (AU)

(72) Inventor: Marcus Patrick Carey, Parkville (AU)

(21) Appl. No.: 14/563,194

(22) Filed: Dec. 8, 2014

(30) Foreign Application Priority Data
Dec. 12, 2013 (AU) 2013904853

(51) Int. Cl. A61F 2/00; A61B 17/42

(52) U.S. Cl. CPC A61F 2/0063; A61B 17/4241

(57) ABSTRACT

A prosthetic device for supporting one or both of the anterior cervix and the anterior vaginal wall in a subject comprises a main body and a pair of flexible arms extending from the main body. At least part of the main body is adapted for attachment to the upper anterior vaginal wall, and the arms are configured for secure attachment to one or more stable body portions to suspendingly support the anterior cervix and anterior vaginal wall when in use.

### TECHNICAL FIELD

The present invention relates to surgical methods to conserve and re-support the prolapsed uterus in women. It relates particularly but not exclusively to methods that may be performed laparoscopically using conventional techniques or with robotic support although open surgical approaches are also contemplated. The invention also relates to devices for use with the surgical procedure.

### BACKGROUND OF INVENTION

Vaginal prolapse is a condition in which the bladder, uterus and/or bowel protrude into the vagina, typically due to loss of natural support for the pelvic organs and the vaginal vault in women who had undergone a prior hysterectomy. In the normal female anatomy, direct support for the vaginal vault is provided by the parametrium (cardinal and uterosacral ligaments) and paracolpium fibers. These fibers act like suspensory ligaments and arise from the fascia of the piriformis muscle, sacroiliac joint and lateral sacrum, and insert into the lateral upper third of the vagina. Indirect support for the vaginal vault is provided by the levator plate, formed by the fusion of the right and left levator ani muscles between the rectum and coccyx. Pelvic organ prolapse and vaginal vault prolapse occurs after failure of these direct and indirect supporting mechanisms and is frequently accompanied by weakness of the muscular pelvic floor and suspensory fibers of the parametrium and upper paracolpium.

In developed countries around one in nine women undergo surgery for pelvic organ prolapse. In the United Sates, more than 400,000 operations for prolapse are performed on over 300,000 women annually for pelvic organ prolapse. Anterior and/or posterior colporrhaphy (native tissue repair) are the most commonly performed operations for pelvic organ prolapse. Sacral colpopexy is widely considered to be the gold standard operation for pelvic organ prolapse particularly in cases of recurrent vaginal prolapse and prolapse of the vaginal vault following hysterectomy. Vaginal vault prolapse occurs in approximately 10% of women following hysterectomy and occurs in equal numbers following abdominal and vaginal hysterectomy. In 2010, around 13% of women having surgery for pelvic organ prolapse in the United States underwent a sacral colpopexy procedure. The sacral colpopexy procedure can be performed through a laparotomy incision, laparoscopically or robotically.

Prolapse of the uterus has traditionally been managed by vaginal hysterectomy and usually performed concomitantly with some form of vaginal surgery to re-support the vaginal apex (e.g. sacrospinous ligament fixation, transvaginal utero sacral ligament suspension). Up to 44% of women undergoing prolapse surgery have a concomitant hysterectomy. However, many patients with uterine prolapse are now requesting conservation of the uterus at the time of prolapse surgery. Many different vaginal, abdominal and laparoscopic procedures have been described to treat pelvic organ prolapse and there is currently no consensus on the most effective operation.

Previously, dissatisfaction with native tissue repair (traditional colporrhaphy) for pelvic organ prolapse resulted in increased usage of mesh to augment vaginal repair procedures in order to obtain higher success rates. However, the use of mesh placed via a trans-vaginal incision during vaginal repair procedures is controversial. Studies have reported significant problems (e.g. pain, dyspareunia and mesh exposure) with the use of mesh during vaginal prolapse surgery. As a result of recent FDA (Food and Drug Administration) warnings about the usage of trans-vaginal mesh there has been renewed interest in native tissue repair, sacral hysteropexy and sacral colpopexy.

Typically the sacral hysteropexy operation involves the attachment of a synthetic or biological graft to the posterior aspect of the cervix and the sacral promontory thereby suspending the uterus from the sacral promontory. Surgeons have also described extension of the synthetic or biological graft onto the posterior vaginal wall. There are numerous problems associated with these current approaches to sacral hysteropexy.

When synthetic or biological grafts are attached only to the posterior cervix and/or upper posterior vagina there is a risk that the graft may become detached from the posterior cervix resulting in failure of surgery and recurrent uterine prolapse. The focus of current methods of sacral hysteropexy is often attachment of the synthetic or biological graft to the posterior cervix resulting in anti-version and anti-flexion of the uterus. In some cases this may result in protrusion of the anterior aspect of the cervix and upper anterior vagina into the vaginal cavity. This results in patients experiencing symptoms of recurrent vaginal bulge (i.e. symptoms consistent with recurrent vaginal prolapse).

It would be desirable to provide an alternative approach to sacral hysteropexy which avoids or at least reduces these problems.

The discussion of the background to the invention included herein including reference to documents, acts, materials, devices, articles and the like is intended to explain the context of the present invention. This is not to be taken as an admission or a suggestion that any of the material referred to was published, known or part of the common general knowledge as at the priority date of any of the claims.

### SUMMARY OF INVENTION

Viewed from one aspect, the present invention provides a prosthetic device for supporting one or both of the anterior cervix and the anterior vaginal wall in a subject, the device comprising a main body and a pair of flexible arms extending from the main body, wherein at least part of the main body is adapted for attachment to the upper anterior vaginal wall, and wherein the arms are configured for secure attachment to one or more stable body portions to suspendingly support the anterior cervix and anterior vaginal wall when in use.

In a preferred embodiment, the main body and aspects of the arms proximal to the main body define a concavity for receiving at least part of the anterior cervix when in use. The concavity is typically defined at least in part by an inner edge of the main body.

Preferably the prosthetic device is a unitary graft although it may alternatively comprise a main body portion to which the arms are attached in a multi-piece construction. Ideally, the prosthetic device has a generally U-shaped appearance, wherein the base of the U comprises the main body. In some embodiments, one or both of the arms includes a contoured inner edge proximal to the main body to receive at least part of the anterior cervix. Thus, the prosthetic device may have an internal “keyhole shape”.

A prosthetic device according to any one of the preceding claims, wherein one or both arms are adapted for insertion through an opening formed in the broad ligament of the uterus of the subject.

One or both of the arms are ideally adapted for attachment at a distal aspect thereof to the anterior longitudinal ligament on the sacral promontory of the subject although in some cases, the arms may be adapted for attachment at a distal aspect thereof to the right and/or left uterosacral ligament. Attachment may be by e.g. sutures, staples, tissue adhesive, surgical clips, screws, tacks, anchors or the like, or by tissue ingrowth after a period of support while the distal ends of the arms are in abutment with the ligament.

Preferably, the prosthetic device is adapted for attachment at one or more locations on the main body to one or both of the anterior cervix and the upper anterior vaginal wall. Attachment may be by e.g. sutures, staples, tissue adhesive, surgical clips, screws, tacks, anchors or the like.

The prosthetic device according may be adapted for insertion into the pelvis through an open abdominal incision, or using a laparoscopic instrument employing conventional or robotic assisted laparoscopic techniques.

Viewed from another aspect, the present invention provides a device for manipulating utero-vaginal anatomy of a subject, the device including: a shaft; a positioning body arranged distally on the shaft; a manipulating member movable along a length of the shaft; and retaining means for retaining the manipulating member at a location along the shaft which is suitable for achieving manipulation of the utero-vaginal anatomy of the subject; wherein the location of the manipulating member along the shaft is selectable according to the subject's own anatomy.

Preferably, the shaft is curved and substantially rigid to assist with manipulation of the utero-vaginal anatomy during surgery. In some embodiments, the shaft is conformable and may be bent to suit the anatomy of an individual subject. A handle may also be provided toward a proximal end of the shaft.

Preferably, the positioning body is an inflatable member, such as a balloon, adapted to be delivered in a deflated condition into the uterine cavity and inflated when inside the uterine cavity to maintain positioning of the device in situ. It is to be understood, however, that other positioning bodies such as collapsibly expandable atraumatic frameworks and baskets may be utilised to position the device in situ.

Similarly, the retaining means may take any suitable form. In some embodiments, the retaining means is inherent in the manipulating member which includes an internal thread adapted to couple with a thread on the shaft, or a collet mechanism, for engaging the manipulating member on the shaft when appropriately positioned. In a preferred embodiment, the retaining means includes: a first retaining collar movably located on the shaft distally of the manipulating member and proximally of the retaining body; and a second retaining collar movably located on the shaft proximally of the manipulating member; wherein the each of the first and second retaining collars is releasably engageable with the shaft to prevent movement along the shaft.

In some embodiments, the first retaining collar is positionable on the shaft such that when engaged with the shaft, the extent to which the shaft may be inserted into the uterus is limited.

Preferably, the manipulating device is configurable so that when in use: the first retaining collar is engaged with the shaft at a distance from a distal tip of the shaft that corresponds to the subject's uterine cavity length; the positioning body is an inflatable member that is inflatable within the uterine cavity; the manipulating member is positioned on the shaft to occupy the upper vagina of the subject; and the second retaining collar is engaged on the shaft to preclude movement of the manipulating member proximally along the shaft. Typically, the manipulating member is positioned in abutment with the first collar at a distal end and in abutment with the second collar at a proximal end.

Viewed from another aspect, the present invention provides a surgical method for supporting a prolapsed uterus in a subject, the method including the steps of: orienting a manipulating device in the upper vagina to facilitate tissue reflection; reflecting the peritoneum off one or both of the anterior cervix and upper anterior vaginal wall; introducing a prosthetic device into the pelvis, the prosthetic device comprising a main body adapted to support one or both of the anterior cervix and the anterior vaginal wall, and a pair of flexible arms extending from the main body; arranging the prosthetic device with the anterior cervix received in a concavity of the prosthetic device defined by the main body and proximal aspects of the arms; and manipulating the arms for secure attachment of a distal aspect of each arm to a stable body portion.

Preferably, arranging the prosthetic device includes arranging at least part of the main body on the upper anterior vaginal wall.

Preferably, the method includes forming an opening in each broad ligament lateral to the uterus, wherein the manipulating step includes drawing an arm of the prosthetic device through a respective opening in the broad ligament for secure attachment of a distal aspect of each arm to the stable body portion.

Preferably, the method also includes securing at least part of the prosthetic device to one or both of the anterior cervix and the upper anterior vaginal wall by e.g. sutures, staples, tissue adhesive, surgical clips, screws, tacks, anchors or the like.

Typically, the stable body portion is the sacral promontory although in some embodiments the right and/or left uterosacral ligament may be used. The method may include the steps of forming an opening in the peritoneum anterior to the sacral promontory and locating a distal aspect of each of the arms of the prosthetic device within the peritoneal opening.

In some embodiments, the peritoneal opening is a tunnel, and the method includes drawing at least a distal end of the arms through the tunnel and into abutment with the anterior longitudinal ligament on the sacral promontory.

Preferably, the method including the step of fixing the distal end of the arms to the anterior longitudinal ligament by e.g. sutures, staples, tissue adhesive, surgical clips, screws, tacks, anchors or the like. In some embodiments, the method includes removing the manipulating device and positioning a vaginal splinting appliance in the vagina to stabilise placement of the prosthetic device for a duration sufficient to achieve tissue repair.

It is to be understood that the surgical method may be performed through an open abdominal incision or using conventional laparoscopy or using robotic-assisted laparoscopy.

Viewed from another aspect, the present invention provides a prosthetic device for supporting a pelvic organ in a subject, the device comprising a main body adapted to seatingly support at least part of the pelvic organ when in use, and a pair of flexible arms extending from the main body, wherein the arms are configured for secure attachment to one or more stable body portions of the subject such that when in use, the attached arms draw the main body of the prosthetic device toward the one or more stable body portions to suspend and support the pelvic organ.

In a preferred embodiment, the prosthetic device comprises a sling adapted to suspendingly support the pelvic organ when the arms are attached to the one or more stable body portions.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

### BRIEF DESCRIPTION OF DRAWINGS

The present invention will now be described in greater detail with reference to the accompanying drawings. It is to be understood that the embodiments shown are examples only and are not to be taken as limiting the scope of the invention as defined in the claims appended hereto.

FIG. 1 is a schematic illustration of a prosthetic device according to an embodiment of the invention.

FIG. 2 is a schematic illustration of a prosthetic device according to another embodiment of the invention.

FIG. 3 is a schematic illustration of a device for manipulating the utero-vaginal anatomy of a subject, according to an embodiment of the invention.

FIG. 4 illustrates use of the manipulating device of FIG. 3 with the balloon inflated in the uterus prior to the manipulating member being positioned in the vagina.

FIG. 5 illustrates the manipulating device of FIGS. 3 and 4 with the manipulating member in the upper vagina and the second collar engaged on the shaft.

FIG. 6 is a schematic illustration showing use of a prosthetic device during a surgical method for repairing a prolapsed uterus, according to an embodiment of the invention.

FIG. 7 is a schematic illustration showing the prosthetic device from FIG. 6 attached to the upper anterior vaginal wall and a vaginal splinting appliance ready for insertion into the vaginal canal.

FIG. 8 is a schematic illustration showing the attached prosthetic device from FIGS. 6 and 7 and a vaginal splinting appliance in the vaginal canal.

### DETAILED DESCRIPTION

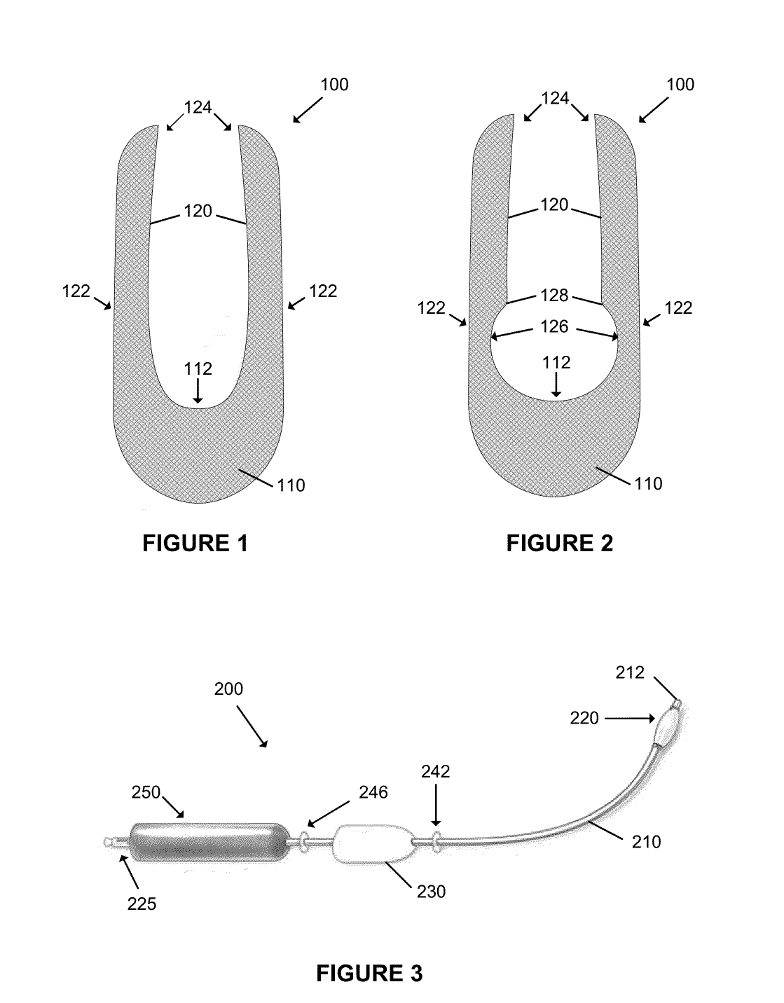

FIGS. 1 and 2 show prosthetic devices 100 for supporting the anterior cervix and the anterior vaginal wall in a subject according to embodiments of the invention. The device has a main body 110 and flexible arms 120 extending from the main body. In use, the main body 110 is, at least in part, attached to the upper anterior vaginal wall and the anterior cervix is received within concavity 112 defined by the main body and the proximal aspects 122 of the arms 120. Arms 120 are attachable to a stable body portion, such as may be found at and around the sacral promontory. When attached, the cervix and upper vagina are suspended and supported, thereby treating the prolapsed condition.

In the embodiments shown, the prosthetic device has a generally U-shape. In the embodiment shown in FIG. 2, the device 100 has an internal contour defined by narrow portions 126 in proximal aspects 122 of arms 120. Narrow portions 126 end at the main arm portions 122 to form a neck 128 that assists with positioning prosthetic device 100 on the anterior cervix.

In other embodiments not shown, the prosthetic device is a belt or sling which is adapted to receive and support the anterior cervix when the sling arms are attached to the stable body portions. Thus, the prosthetic need not be attached to the anterior vaginal wall. However, where a wider main body portion is provided such as in the illustrated embodiments, attachment to the upper anterior vaginal wall also reinforces the prolapsed vaginal tissue.

The prosthetic device may be formed from a biological material or a synthetic material such as polypropylene or the like, or it may be a hybrid biological/synthetic material. In some embodiments, the prosthetic device s formed from a mesh, laser cut or structured material with interstices or for encouraging tissue ingrowth. The prosthetic device may also have eyelets or other features in the main body 110 for facilitating attachment by sutures, staples, tacks, anchors or other means, to the upper anterior vaginal wall, and/or the anterior cervix. Similarly, eyelets or other features may be provided on distal aspects 124 of the prosthetic device to facilitate attachment to the stable body portion

The prosthetic device of FIGS. 1 and 2 is intended for use in a new surgical approach to treatment of prolapsed uterus (sacral hysteropexy). This method may be performed by conventional laparoscopy, robotic assisted laparoscopy or through an open abdominal incision. In preferred embodiments, the method involves use of a manipulating device 200 of the type illustrated in FIG. 3.

Manipulating device 200 has a shaft 210 which, in a preferred embodiment is rigid and curved to assist with elevation and manipulation of the uterus and vagina. It is to be understood however that the orientation and location of the curve need not be fixed, and that in some embodiments shaft 210 may be re-shaped by the surgeon to suit the anatomy of an individual patient. A positioning body in the form of an inflatable balloon 220 is located distally on shaft 210 and an inflation valve 225 is provided proximally for inflation and deflation of the balloon during use. Manipulating member 230 is movable along a length of shaft 210 and retaining means which, in the form of movable collars 242, 246 are releasably engageable with the shaft.

Distal collar 242 when engaged with shaft 210 prevents manipulating member 230 from moving distally along the shaft. When appropriately positioned (see below), distal collar 242 also protects the uterus from perforation by the distal tip 212 of shaft 210. Proximal collar 246 when engaged with shaft 210 prevents manipulating member 230 from moving proximally along the shaft. A handle 250 is provided toward the proximal end of shaft 210. Handle 250 is manipulated by the surgeon to mobilise and position the uterus and/or the upper anterior vagina. Balloon 220 assists with manipulation of the uterus and also retains the position of manipulating device 200 in the vagina and uterus device during surgery.

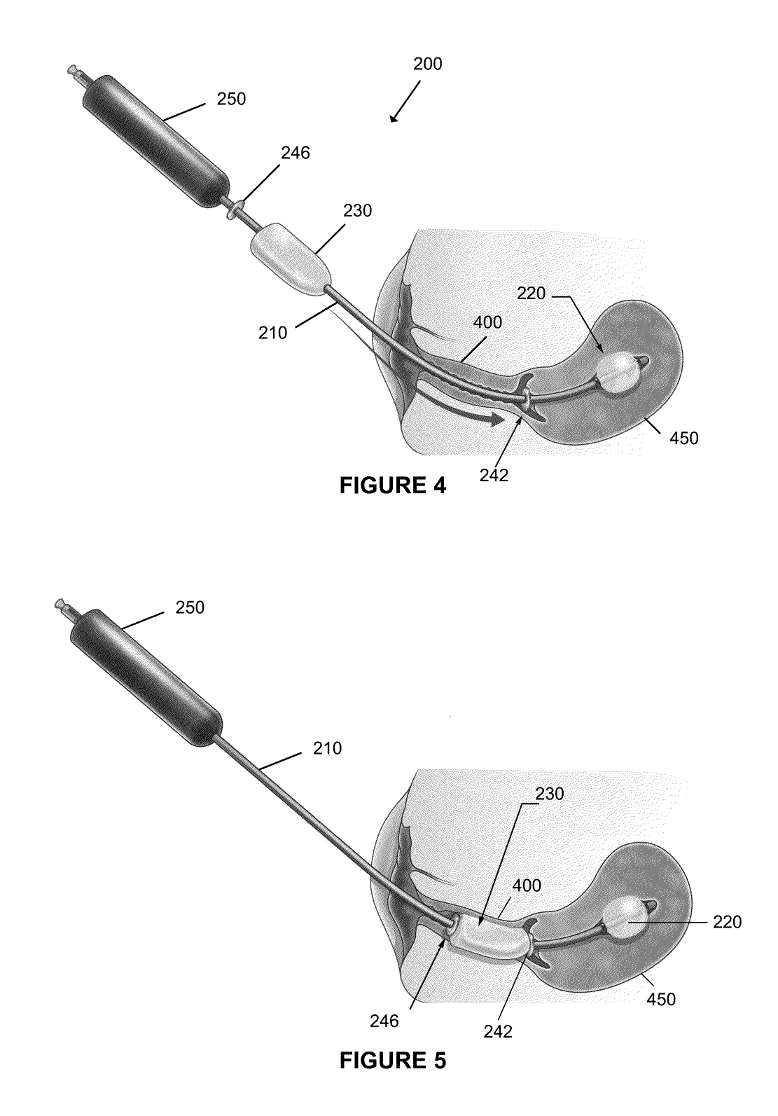

FIGS. 4 to 8 illustrate steps in a surgical method according to an embodiment of the invention, as well as use of the devices disclosed herein.

In a preferred embodiment, a standard surgical instrument is used to grasp and stabilise the anterior cervix. The length of the uterine cavity is determined by a standard uterine sound, as the distance from the external cervical os to the endometrial cavity aspect of the uterine fundus. This length is used to determine the position of distal collar 242 on shaft 210 of manipulating device 200. Thus, prior to insertion of device 200, collar 242 is positioned on and engaged with shaft 210 at a distance from distal tip 212 which is equal to the uterine cavity length.

The cervix is dilated with standard cervical dilators known in the art and the device 200 is inserted into the uterine cavity until first collar 242 is in abutment with the external cervical os. This prevents over-insertion of the device into uterus 450 limiting the risk of perforating the uterus with distal tip 212 of shaft 210. It may also be desirable to provide an atraumatic coating on distal tip 212 to further reduce risk of perforation. With device 200 inserted, balloon 220 is inflated typically with air, by using a syringe or other suitable inflation fluid source coupled via inflation valve 225. Inflated balloon 220 inside uterus 450 retains the position of manipulating device 200 during the procedure. The surgical instrument used to grasp and stabilise the anterior cervix is then removed. FIG. 4 illustrates manipulating device 200 with balloon 220 inflated in uterus 450, prior to positioning manipulating member 230 in vagina 400.

With shaft 210 and inflated balloon 220 in position, manipulating member 230 is advanced along shaft 210 and moved into the upper vagina, typically into abutment with distal collar 242 which precludes further advancement of the manipulating member distally along the shaft. Proximal collar 246 is advanced distally to abut the proximal end of manipulating member 230 where it is engaged with shaft 210 to prevent movement of the manipulating member proximally along the shaft during utero-vaginal manipulation and surgical dissection. FIG. 5 illustrates manipulating device 200 with manipulating member 230 in the upper vagina and second collar 246 engaged on shaft 210.

With the aid of the manipulating device 200 which is oriented by the surgeon manipulating handle 250, the uterus is placed in an axial and cranial position. The peritoneum anterior to the cervix is dissected transversely and reflected off the anterior cervix and upper anterior vaginal wall. The bladder is then reflected off the upper anterior vaginal wall. Using manipulating device 200 to position inflated balloon 220, the uterus is ante-verted and an openings 552, 556 are created in the broad uterine ligament (not shown) on each side lateral to the uterus 450 and above each uterine artery (not shown).

The peritoneum over the sacral promontory is dissected to reveal the anterior longitudinal ligament 560 on the sacral promontory. In one embodiment a tunnel (not shown) is created from the peritoneum incision made over the sacral promontory in the midline to a point approximately 3 to 6 cm below the sacral promontory. In another embodiment the peritoneum is dissected from the sacral promontory to the cul-de-sac and posterior cervix. After the requisite openings are formed for receiving distal aspects 124 of prosthetic device arms 120, the device itself is introduced into the pelvis. FIGS. 1 and 2 illustrate examples of prosthetic devices.

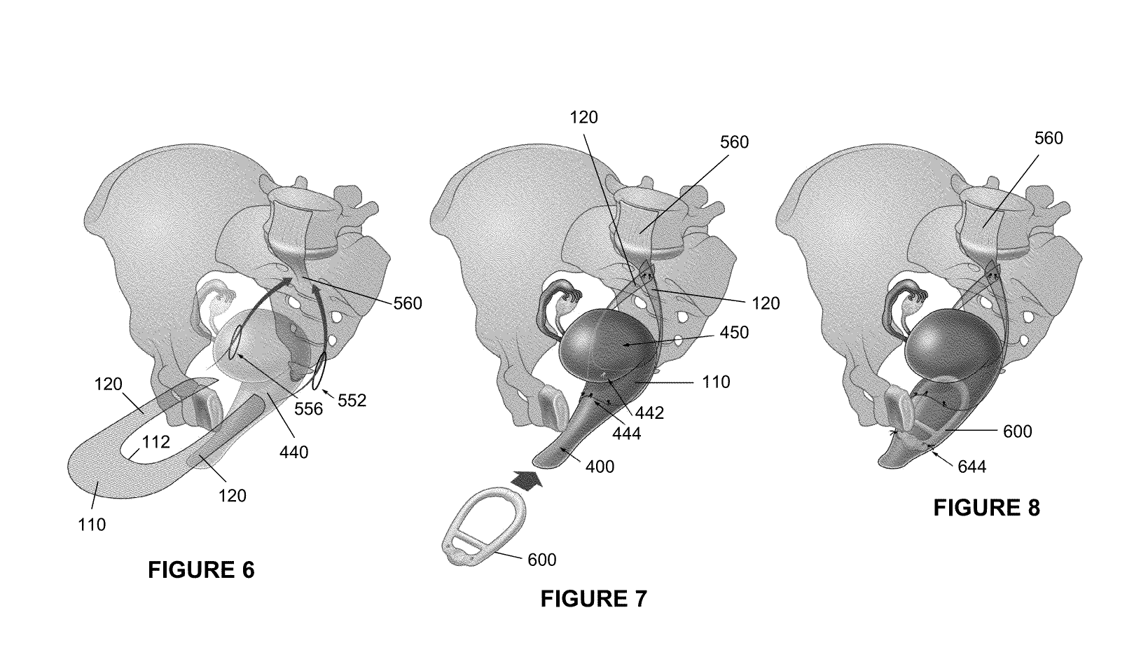

Prosthetic device 100 may be rolled, folded, wrapped or otherwise minimised in overall dimension for delivery to the pelvis through laparoscopic instruments, or by an open abdominal incision. When delivered to the implantation site, the prosthetic is unravelled or otherwise assumes its normal shape which, in preferred embodiments, is a generally U-shape. FIG. 6 shows prosthetic device 100 ready for placement.

Concavity 112 of prosthetic device 100 is placed on the anterior cervix and part of main body 110 is placed on upper anterior vaginal wall 440. In some embodiments prosthetic device 100 is sutured to the anterior cervix (see suture 442 represented in “transparent view” in FIG. 7) and/or sutured to upper anterior vaginal wall 440 using sutures 444 (FIG. 7). Additionally/alternatively, prosthetic device 100 may be secured to the upper anterior vaginal wall 440 and/or anterior cervix using one or more alternative methods such as tissue adhesive, surgical clips, screws tacks, anchors or the like. Arms 120 of prosthetic device 100 are then drawn through respective broad ligament windows 552, 556 for attachment to a stable body portion, typically tissue near the sacral promontory.

In some embodiments, a tunnel (not shown) is created under the peritoneum extending in an inferior direction from the sacral promontory and instruments are used to draw prosthetic device arms 120 through the tunnel so that distal aspects 124 of the arms abut anterior longitudinal ligament 560. Arms 120 may be fixed to anterior longitudinal ligament 560 sutures, staples, tissue adhesive, surgical clips, screws, tacks, anchors or the like. Alternatively arms 120 may abut or rest in contact with anterior longitudinal ligament 560 on the sacral promontory without fixation. In the latter scenario, prosthetic device 100 requires stabilisation in situ for a duration long enough to achieve tissue growth on or into the prosthetic to form an attachment at anterior longitudinal ligament 560 which provides the necessary support to the vagina and uterus.

In some embodiments, the peritoneum is dissected from the sacral promontory to posterior cervix and distal aspects of prosthetic device arms 120 are attached to the anterior longitudinal ligament on the sacral promontory by sutures, staples, tissue adhesive, surgical clips, screws, tacks, anchors or the like.

In clinical situations where it is not safe or surgically feasible to access the sacral promontory, the surgeon may choose instead to attach the distal aspects of prosthetic arms 120 to one or both of the right and left uterosacral ligament (not shown). Attachment may be e.g. by sutures, staples, tissue adhesive, surgical clips, screws, tacks, anchors or the like. Alternatively/additionally, the surgeon may allow distal aspects of prosthetic arms 120 to rest in abutment with the right and/or left uterosacral ligament without fixation. This scenario requires stabilisation of the prosthetic device 100 in situ for a duration long enough to achieve tissue growth on or into the prosthetic to form an attachment at the uterosacral ligament which provides the necessary support to the vagina and uterus.

At the surgeon's discretion the peritoneum may be closed over, or approximated to, prosthetic device 100. Alternatively the surgeon may choose not to close the peritoneum over prosthetic device 100 or approximate the peritoneum to the edge of the prosthetic device.

At the completion of surgery, balloon 220 of manipulating device 200 is deflated by opening inflation valve 225 and the manipulating device is retracted from the uterus and vagina. Preferably, a splinting appliance is placed into the vagina and remains in place for 3 to 4 weeks following surgery. Splinting appliances and their use in treating pelvic organ prolapse are described in application PCT/AU2013/000415 filed 24 Apr. 2013, and application PCT/AU2014/000027 filed 16 Jan. 2014, the entire contents of both of which are hereby incorporated herein by reference.

Following surgery for pelvic organ prolapse, the repaired tissues are exposed to rises in intra-abdominal pressure as the patient mobilizes or with coughing, vomiting and straining with bowel evacuation. Rises in intra-abdominal pressure may adversely affect healing leading to surgical failure and recurrent prolapse. By supporting the vagina with a splinting appliance for a minimum of 25 days following surgery the risk of surgical failure and recurrent pelvic organ prolapse may be reduced. Furthermore the use of a vaginal splinting appliance following surgery is likely to reduce the need for multiple sutures, staples or anchoring devices in order to secure the synthetic or biological graft onto the vaginal and cervical tissues and anterior longitudinal ligament on the sacral promontory. Advantageously, this is likely to reduce the complexity of surgery and reduce the potential complications associated with the use of multiple sutures, staples or anchoring devices on the vaginal and cervical tissues and the anterior longitudinal ligament on the sacral promontory. FIG. 7 shows prosthetic device 100 attached to upper anterior vaginal wall 440 and anterior longitudinal ligament 560 with vaginal splinting appliance 600 ready for insertion into vagina 400. FIG. 8 shows vaginal splinting appliance 600 secured in vagina 400 by removable/restorable sutures 644.

To the inventor's knowledge, a vaginal support device to splint the healing vaginal tissues has not previously been used or studied as an adjunct to sacral hysteropexy. If concomitant transvaginal prolapse surgery is performed it is desirable that a tamponade be placed in the vaginal cavity at the completion of surgery, in addition to the surgical vaginal support.

Advantageously, the inventive methods and devices involve placement of a prosthetic device onto the anterior cervix, rather than the posterior cervix, as is the case in traditional sacral hysteropexy. This novel approach provides a sling arrangement in which the anterior cervix and upper anterior vaginal wall are, at least in part, seatingly supported when suspended from the sacral promontory. The sling arrangement extending around the utero-vaginal tissue reduces the likelihood of the prosthetic becoming detached having obvious beneficial impacts on surgical outcomes.

Additionally, the novel approach disclosed herein is likely to result in the uterus obtaining a more neutral position post-surgery. This neutral position of the uterus is likely to reduce the risk of patients experiencing the sensation of recurrent uterine and upper anterior vaginal wall prolapse.

It is to be understood that various modifications, additions and/or alterations may be made to the parts previously described without departing from the ambit of the present invention as defined in the claims appended hereto.

1. A prosthetic device for supporting one or both of the anterior cervix and the anterior vaginal wall in a subject, the device comprising a main body and a pair of flexible arms extending from the main body, wherein at least part of the main body is adapted for attachment to the upper anterior vaginal wall, and wherein the arms are configured for secure attachment to one or more stable body portions to suspendingly support the anterior cervix and anterior vaginal wall when in use.

2. A prosthetic device according to claim 1, wherein the main body and proximal aspects of the arms define a concavity for receiving at least part of the anterior cervix when in use.

3. A prosthetic device according to claim 2, wherein the concavity is defined at least in part by an inner edge of the main body.

4. A prosthetic device according to claim 1, wherein the device is a unitary graft having a generally U-shaped appearance, wherein the base of the U comprises the main body.

5. A prosthetic device according to claim 1, wherein at least one of the arms includes a contoured inner edge proximal to the main body to receive at least part of the anterior cervix.

6. A prosthetic device according to claim 1, wherein one or both of the arms are adapted for one or more of:
(a) insertion through an opening formed in the broad ligament of the uterus of the subject; and
(b) attachment at a distal aspect thereof to:
(i) the anterior longitudinal ligament on the sacral promontory of the subject; or
(ii) one or both of the right and left uterosacral ligaments.

7. A prosthetic device according to claim 1, adapted for attachment at one or more locations on the main body to one or both of the anterior cervix and the upper anterior vaginal wall.

8. A prosthetic device according to claim 1, adapted for insertion into the pelvis through an open abdominal incision, or using a laparoscopic instrument using conventional or robotic assisted laparoscopic techniques.

9. A method for supporting a prolapsed uterus using the prosthetic device according to any one of the preceding claims.

10. A device for manipulating utero-vaginal anatomy of a subject, the device including:
a. a shaft;
b. a positioning body arranged distally on the shaft;
c. a manipulating member movable along a length of the shaft; and
d. retaining means for retaining the manipulating member at a location along the shaft which is suitable for achieving manipulation of the utero-vaginal anatomy of the subject;
wherein the location of the manipulating member along the shaft is selectable according to the subject's own anatomy.

11. A device according to claim 10, wherein the positioning body is an inflatable member adapted to be delivered in a deflated condition into the uterine cavity of the subject and inflated to maintain positioning of the device in situ.

12. A device according to claim 10, wherein the retaining means includes:
a. a first retaining collar movably located on the shaft distally of the manipulating member and proximally of the retaining body; and
b. a second retaining collar movably located on the shaft proximally of the manipulating member;
wherein the each of the first and second retaining collars is releasably engageable with the shaft to prevent movement along the shaft.

13. A device according to claim 12, wherein the first retaining collar is positionable on the shaft such that when engaged with the shaft, the extent to which the shaft may be inserted into the uterus is limited.

14. A device according to claim 12, being configurable so that when in use:
a. the first retaining collar is engaged with the shaft at a distance from a distal tip of the shaft that corresponds to the subject's uterine cavity length;
b. the positioning body is an inflatable member that is inflatable within the uterine cavity;
c. the manipulating member is positioned on the shaft to occupy the upper vagina of the subject; and
d. the second retaining collar is engaged on the shaft to preclude movement of the manipulating member proximally along the shaft.

15. A device according to claim 14 wherein, when in use, the manipulating member is positioned in abutment with the first collar at a distal end and in abutment with the second collar at a proximal end.

16. A device according to claim 10, including one or more of:
(a) the shaft being substantially rigid;
(b) the shaft being curved; and
(c) a proximal handle.

17. A surgical method for supporting a prolapsed uterus in a subject, the method including the steps of:
a. orienting a manipulating device in the upper vagina to facilitate tissue reflection;
b. reflecting the peritoneum off one or both of the anterior cervix and upper anterior vaginal wall;
c. introducing a prosthetic device into the pelvis, the prosthetic device comprising a main body adapted to support one or both of the anterior cervix and the anterior vaginal wall, and a pair of flexible arms extending from the main body;
d. arranging the prosthetic device with the anterior cervix received in a concavity of the prosthetic device defined by the main body and proximal aspects of the arms; and
e. manipulating the arms for secure attachment of a distal aspect of each arm to a stable body portion.

18. A method according to claim 17, wherein arranging the prosthetic device includes arranging at least part of the main body on the upper anterior vaginal wall.

19. A method according to claim 17, including one or more of the steps of:
(a) forming an opening in each broad ligament lateral to the uterus, wherein the manipulating step includes drawing an arm of the prosthetic device through a respective opening in the broad ligament for secure attachment of a distal aspect of each arm to the stable body portion;
(b) securing at least part of the prosthetic device to one or both of the anterior cervix and the upper anterior vaginal wall; and
(c) removing the manipulating device and positioning a vaginal splinting appliance in the vagina to stabilise placement of the prosthetic device for a duration sufficient to achieve tissue repair.

20. A method according to claim 17, wherein the stable body portion is the sacral promontory, and the method includes the steps of forming an opening in the peritoneum anterior to the sacral promontory and locating a distal aspect of each of the arms of the prosthetic device within the peritoneal opening.

21. A method according to claim 20 wherein the peritoneal opening is a tunnel, and the method includes one or more of:
(a) drawing at least a distal end of the arms through the tunnel and into abutment with the anterior longitudinal ligament on the sacral promontory; and
(b) fixing the distal end of the arms to the anterior longitudinal ligament.

22. A method according to claim 17, wherein the stable body portion includes one or both of the right and left uterosacral ligament.

23. A method according to claim 17 including use of one or both of a prosthetic device and a manipulating device;
the prosthetic device comprising a main body and a pair of flexible arms extending from the main body, wherein at least part of the main body is adapted for attachment to the upper anterior vaginal wall, and wherein the arms are configured for secure attachment to one or more stable body portions to suspendingly support the anterior cervix and anterior vaginal wall when in use; and
the manipulating device comprising a shaft, a positioning body arranged distally on the shaft, a manipulating member movable along a length of the shaft, and retaining means for retaining the manipulating member at a location along the shaft which is suitable for achieving manipulation of the utero-vaginal anatomy of the subject, wherein the location of the manipulating member along the shaft is selectable according to the subject's own anatomy.

24. A method according to claim 17, performed through an open abdominal incision or using conventional laparoscopy or using robotic-assisted laparoscopy.

25. A prosthetic device for supporting a pelvic organ in a subject, the device comprising a main body adapted to seatingly support at least part of the pelvic organ when in use, and a pair of flexible arms extending from the main body, wherein the arms are configured for secure attachment to one or more stable body portions of the subject such that when in use, the attached arms draw the main body of the prosthetic device toward the one or more stable body portions to suspend and support the pelvic organ.
